(19) United States
(12) Patent Application Publication
(10) Pub. No.: US 2008/0210628 A1
(43) Pub. Date: Sep. 4, 2008

(54) Process for the Biological Treatment of Ammonium-Rich Aqueous Media

(75) Inventors: Ingo Schmidt, Groesbeek (NL); Michael Silvester Maria Jetten, Nijmegen (NL); Rogier Nicolaas Antonius Van Kempen, Wageningen (NL)

Correspondence Address: TROUTMAN SANDERS LLP, 600 PEACHTREE STREET , NE, ATLANTA, GA, 30308, US

(73) Assignees: Grontmij Advies & Techniek B.V., De Bilt (NL); Stichting Voor DeTechnische Wetenschappen, Utrecht (NL); Stichting Katholieke Universiteit Genoemd Radboud Universiteit Nijmegen, Nijmegen (NL)

(21) Appl. No.: 10/556,692

(22) PCT Filed: May 16, 2003
(86) PCT No.: PCT/NL03/00360

§ 371 Date: Oct. 10, 2006

(51) Int. Cl. C02F 3/34; C12N 1/20

(52) U.S. Cl. 210/610; 435/252.1; 435/262.5

(57) ABSTRACT

The invention relates to a process for the biological treatment of ammonium-rich aqueous media in the presence of ammonia oxidizing bacteria, by supplying nitrogen dioxide (NO₂) into said aqueous media, thus effecting a nitrification and a denitrification, wherein further nitrogen oxide (NO) is supplied into said media such that the ratio of NO:NO₂ is in the range from 1:2 to 1:500 (v/v). Further the use of off-gases in this process is disclosed as well as microorganisms cultured in the presence of NO and NO₂.

The invention relates to a process for the biological treatment of ammonium-rich aqueous media.

More specifically, is the invention directed to a process for the biological treatment of ammonium-rich aqueous media in the presence of ammonia oxidizing bacteria by supplying gaseous nitrogen dioxide into said aqueous media, thus effecting nitrification and denitrification processes.

It is observed that it is common knowledge that ammonia oxidizing bacteria, for example organisms of the genera Nitrosomonas, can be used to remove ammonia dissolved in water, and wastewater especially. The ammonia oxidation which takes place in a first step in such a process is restricted to oxic conditions, wherein molecular oxygen is used as the oxidizing compound to form nitrite. In a second step, a different group of micro-organisms, such as the nitrite oxidizing Nitrobacter, oxidize nitrite further to nitrate under oxic conditions. Since the formation of nitrite and nitrate is not the aim of the biological treatment of ammonia in wastewater, a further step called denitrification is necessary to convert nitrite and/or nitrate into gaseous molecular di-nitrogen (N₂). The denitrification usually takes place under anoxic conditions in one (anoxic niches e.g. in flocs) or in separated compartments. The organisms catalyzing this reaction, called denitrifiers, need organic compounds, e.g. already available in the wastewater or an external carbon source such as methanol. Such a process is for example disclosed in EP-B-0 826 639.

Since the formation of nitrate requires about 25% more oxygen than the formation of nitrite and the formation of N₂ from nitrate requires about 40% more of the organic compounds than the conversion of nitrite, the aim in wastewater treatment is nowadays to prevent the nitrate formation by the nitrite oxidizing micro-organisms in the nitrification step.

A process as has been indicated in the preamble is known from DE-C-19 617 331. Nitrogen dioxide is according to this known process used as an oxidant for ammonium being present in the aqueous media. It is stated that the advantage of the process is to be estimated in the fact that the reduction equivalents formed in the oxidation of the hydroxylamine by the addition of nitrogen dioxide must not be used again, partly, for the oxidation of the ammonia, but will be available for the reduction of the formed nitrite.

Further, it is stated that when the oxidation of ammonia is effected with externally supplied nitrogen dioxide, instead of oxygen, no reduction equivalents will be consumed, so that the reduction equivalents produced in the conversion of hydroxylamine into nitrite, can be used for energy generation.

Further research of this process showed that the assumptions made in DE-C-19 617 331 are incorrect, with the result that the technical realization of the process can not be repeated easily by a skilled person.

During said research it was now surprisingly found that it is possible to control the metabolic activity of ammonia oxidizing bacteria via both the nitrogen monoxide (NO) and nitrogen dioxide (NO₂) concentrations in the nitrification step, more specifically by maintaining a certain ratio between NO and NO₂ in the nitrification step.

The present invention is directed to this finding and thus relates to a process as indicated above, which process is characterized in that further nitrogen monoxide (NO) is supplied into said media, such that the ratio of NO:NO₂ is from 1:2 to 1:500 (v/v).

The present process thus enables the realization of combined ammonia, nitrite and NOₓ removal from wastewater and off-gases by controlling the NO/NO₂ ratio. This could be obtained due to the fact that the metabolic activity of ammonia oxidizing bacteria could be controlled, leading to an optimization of the treatment process, i.e. an activation of nitrite denitrification and an (considerable) inactivation of nitrite oxidation by the nitrite oxidizing bacteria (hereinafter sometimes called “nitrite oxidizers”).

Preferably the NO/NO₂ ratio in said aqueous media is from 1:2 to 1:100, more preferably from 1:2 to 1:25, more specifically 1:3 (v/v).

Due to the addition of NOₓ, several metabolic activities of ammonia oxidizing micro-organisms (hereinafter called “ammonia oxidizers) are influenced, and the process of nitrogen removal can be optimized and accelerated. In the presence of NO₂, ammonia oxidizers use this additional oxidant for their ammonia oxidation (eq. 1). In comparison with the ammonia oxidation with oxygen as oxidant (ΔG^{0,}−120 kJ mol⁻¹) the oxidation with NO₂ (ΔG^{0,}−140 kJ mol⁻¹) is energetically more efficient. The ammonia oxidation is accelerated in the presence of NO₂. Consequently, more ammonia can be consumed per unit of time and per unit of volume leading to an optimized treatment of wastewater containing ammonia. The oxidation of hydroxylamine into nitrite is in agreement with earlier findings (eq. 2).

NH₃+2 NO₂+2H⁺+2e⁻→NH₂OH+H₂O+2 NO ΔG^{0,}−140 kJ mol⁻¹ (1)

NH₂OH+H₂O→HNO₂+4H⁺+4e⁻ΔG^{0,}−289 kJ mol⁻¹ (2)

2 NO+O₂→2 NO₂ (3)

HNO₂+3H⁺+3e⁻→0.5 N₂+2 H₂O (4)

Surprisingly, ammonia oxidizers now appeared to be able to catalyze the (re)oxidation of NO, a product of the ammonia oxidation with NO₂ (eq. 1), to NO₂ (eq. 3). Since the ammonia oxidizers are able to regenerate the oxidant NO₂ from NO, it is not necessary to add NO₂ in a 1:2 stoichiometric according to ammonia (eq. 1). A NO₂/ammonia ratio of 1:200 to 1:2,000 could be shown to be sufficient. Optimal results can be established at a ratio of about 1:500.

According to an expedient embodiment of the present process is the NO₂ concentration in the aqueous media regulated between 1 and 500 ppm (v/v), preferably 25-250 ppm (v/v), more specifically 75 ppm (v/v).

The NO concentration, on the other hand, is expediently established between 1 and 100 ppm (v/v), preferably 10-50 ppm (v/v), more specifically 25 ppm (v/v).

Another surprising result of the present process was the influence of NO on the metabolic activity of the ammonia oxidizers, since No is known as a toxic compound for many micro-organisms (1 ppm v/v is already lethal for many micro-organisms). It now nevertheless appeared that ammonia oxidizers are highly resistant to NO and tolerate concentrations of more than 500 ppm v/v. Furthermore, it was surprising that the metabolic activity of ammonia oxidizers could be controlled via the NO concentration.

Although applicant does not wish to be bound to any theory, it is assumed that nitrogen monoxide (NO) is responsible for different processes:

a) NO can be used to induce a denitrification activity of ammonia oxidizers. As a consequence, ammonia oxidizers start to consume nitrite under fully oxic conditions. Therefore, a combined nitrification and partial denitrification (up to 66%) is possible catalyzed by only one group of bacteria (aerobic ammonia oxidizers) in a one step system;

b) The denitrification activity of the ammonia oxidizers is independent of organic compounds. The organic compounds necessary in the classical denitrification are here replaced by ammonia. The effect of NO reduces the total need of organic compounds. If the COD of the wastewater is not sufficient for the removal of the remaining nitrite (not denitrified during ammonia oxidation), less (up to 40%) organic compounds have to be added in the classical anoxic denitrification step (pre-denitrification, post-denitrification);

c) In contrast to the ammonia oxidizers the nitrite oxidizers are highly sensitive against NO. When NO is added, their activity is repressed and their cell number decreases; hardly any nitrate is formed in the nitrification step. Therefore, besides gaseous molecular di-nitrogen (N₂), nitrite is the main product of the nitrification step. Since mainly nitrite has to be converted into N₂, the consumption of organic compounds in the denitrification step is reduced (up to 40%). The total amount of organic compounds can be reduced up to 80% compared to classical nitrification/denitrification via nitrate. This reduction can result in a reduction (up to 80%) of the amount of surplus sludge produced compared to the classical nitrification/denitrification process. When less biosludge is formed the sludge retention time (SRT) of an existing nitrifying/denitrifying wastewater treatment plant can be increased, if the excess of organic compounds is removed by prior treatment such as pre-settling and preceding aeration. Since the maximum nitrogen treatment capacity of a treatment plant is often limited by the SRT, a reduced production of activated sludge and a subsequently increased SRT can increase the nitrogen treatment capacity of a treatment plant. Furthermore, the basins for N removal of new plants can be designed and constructed significantly (up to 80%, typically 50%) smaller.

Although the present process can thus, as usual, be executed without sludge retention, it is possible to execute the process, according to a preferred embodiment, with sludge retention.

It was further surprisingly found that the supply of NO/NO₂ into the ammonium-rich aqueous media resulted into a considerable reduction (up to 50%) of the NO and NO₂ concentration in the off-gas of the nitrification step. This is due to the fact that the nitrification activity of the ammonia oxidizers is optimized and therefore less NO is produced. Additionally, the denitrifying bacteria (also contributing to the emission of greenhouse gasses) are inhibited (inactive) in the presence of NO. These off-gases could nevertheless be used for the determination of the amount of NO/NO₂ gases needed in the present process.

The invention thus relates according to a preferred embodiment to a process as indicated before, wherein the amount of NO/NO₂ gases needed in the biological treatment is controlled as a function of the NOₓ emission from the nitrification step.

Although the NO and NO₂ gases needed in the present process can be supplied from any source, it is preferred to use off-gases produced by combustion of fossil fuels (engines) or from heat power plants.

It was further surprisingly found that the addition of NO leads to a structure change of the activated sludge flocs. In the presence of NO, the micro-organisms form very compact flocs with improved settling and filtration characteristics.

The invention therefore also relates to micro-organisms, especially of the genera Nitrosomonas, which have been cultured in the presence of NO and NO₂.

It was also observed that the growth rate of the non-nitrifying bacteria and nitrite oxidizers, as used in the present process, is reduced in the presence of NO. Hence, less activated sludge with a higher content of ammonia oxidizers is formed which of course provides a higher specific ammonia oxidation activity.

The invention thus also relates to the use of micro-organisms of the genera Nitrosomonas, cultured in the presence of nitrogen dioxide and nitrogen monoxide, as activated sludge in a process for the biological treatment of ammonium-rich aqueous media.

As a consequence of the above advantages, biomass retention by a separation unit such as a settling tank, a lamellae separator or a membrane system can easily be optimized. Up to 50% smaller settling tanks, lamellae separators and higher membrane fluxes are feasible.

The invention will now be explained by means of an example, representing a preferred embodiment of the process according to the invention, with reference to the drawings, wherein

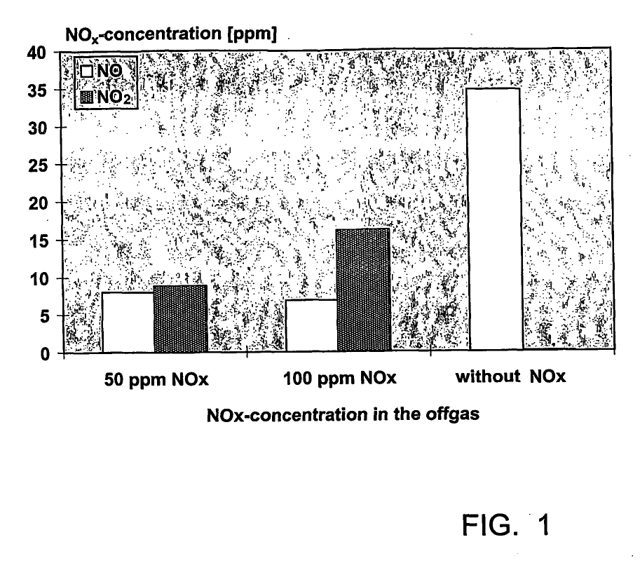

FIG. 1 shows the effects of the NO/NO₂ supply to the NOₓ concentration in the off gas, and

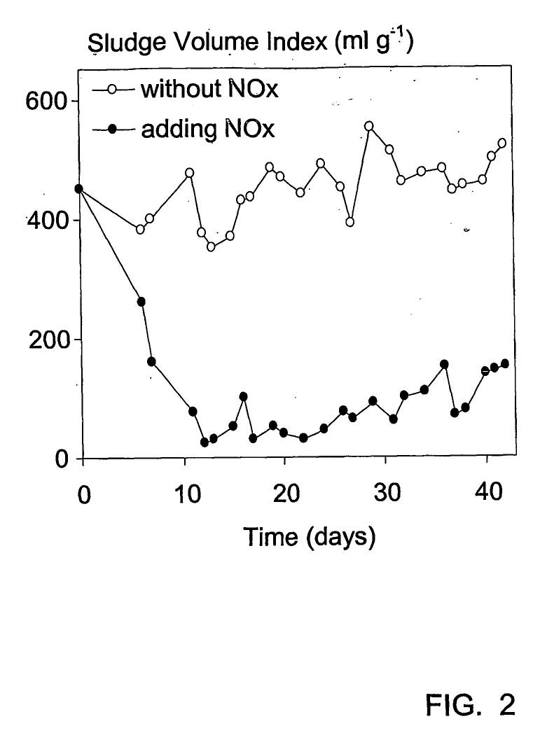

FIG. 2 shows the effect of the NOₓ supply to the sludge volume index.

### EXAMPLE

Biomass (activated sludge) was grown in 5 l laboratory scale reactors with 3.5 l medium without biomass retention. The reactor was aerated (0.1 to 2 l min⁻¹) with variable mixtures of compressed air, argon, NO₂ (0 to 500 ppm), and NO (0 to 500 ppm) using four mass-flow controllers. NO and NO₂ concentrations in the fresh (inlet) and off-gas (outlet) were permanently measured and level, temperature, dissolved oxygen (DO) and pH-value were measured and controlled regularly. Temperature was maintained at 22° C., DO at 0.04-6 mg l⁻¹. The pH-value was kept at 7.4 by means of a 20% Na₂CO₃-solution. Samples for offline determination of ammonium, nitrite, nitrate and NOₓ concentrations were taken within regular time intervals. The dilution rate varied between 0.002 (start-up) and 0.1 h⁻¹. The medium contained 150-3,000 mg NH₄⁺-N per liter (10-200 mM NH₄⁺). The effluent was collected and stored at 4° C. for later analytical determinations and analysis of the biomass composition. The reactor was inoculated with 400 ml of activated sludge (B-step). Control experiments were carried out with N. eutropha, cell-free medium and heat-sterilized cell suspensions (activated sludge).

The results of this experiment are represented in FIG. 1,

wherein more specifically the effects of the supply with 0, 50 or 100 ppm NOₓ to the NO and NO₂ concentration in the off-gas of the nitrification step are represented. The NOₓ supplied into the system consisted of NO/NO₂ in a ratio of 1:3 (v/v). FIG. 2 illustrates in a similar experiment (but with sludge retention), the effects of the supply with 0 or 100 ppm NOₓ to the sludge volume index (settling characteristics) of the biomass. It is observed that the sludge volume index (SVI) is a measure for the settling characteristic of the biomass. A lower value of the SVI indicates a more compact or dense biomass with better settling characteristics. The favourable effect of the NO/NO₂ addition in a process for the biological treatment of ammonium-rich aqueous media, on the SVI of the biomass, clearly appears from FIG. 2. The NOₓ concentrations in the off-gas were during both experiments used as a measure of the consumed amount of NOₓ by the system. The difference between the inlet and outlet NOₓ concentrations is used to control the process.

It is observed that although the above described experiments have been executed on a laboratory scale, comparable results were obtained in a pilot plant and thus can also be expected in a full scale wastewater treatment plant.

Further it is observed that after the biological treatment step the biomass and the effluent water can be separated for example with a settler, membranes (e.g. micro or ultra) or dissolved air flotation (DAF) in a biomass separation step. The biomass can be recycled (sludge retention) into the present biological treatment process or removed from the system. If the minimum sludge retention time (SRT) required for nitrification is shorter or equals the hydraulic retention time (HRT), a sludge retention/separation is not necessary. Then, the effluent inclusive the biomass can be directly discharged from the biological treatment process.

1. A process for the biological treatment of ammonium-rich aqueous media in the presence of ammonia oxidizing bacteria, by supplying gaseous nitrogen dioxide (NO₂) into said aqueous media, thus effecting a nitrification and a denitrification process, wherein further nitrogen monoxide (NO) is supplied into said media such that the ratio of NO:NO₂ is in the range from 1:2 to 1:500 (v/v).

2. A process according to claim 1, wherein the NO/NO₂ ratio is from 1:2 to 1:100 (v/v).

3. A process according to claim 1, wherein the ratio of NO₂ to ammonia in said media is in the range from 1:200 to 1:2000 (v/v).

4. A process according to claim 1, wherein the NO₂ concentration in said media is regulated in the range from 1-500 ppm (v/v).

5. A process according to claim 4, wherein the NO₂ concentration in said media is regulated in the range from 25-250 ppm (v/v).

6. A process according to claim 1, wherein the NO concentration in said media is regulated in the range from 1-100 ppm (v/v).

7. A process according to claim 6, wherein the NO concentration in said media is regulated in the range from 10-50 ppm (v/v).

8. A process according to claim 1, wherein the amount of NO/NO₂ gases needed in the biological treatment is controlled as a function of the NOₓ emission from the nitrification step.

9. A process according to claim 1, wherein said process is executed without sludge retention.

10. A process according to claim 1, wherein said process is executed with sludge retention.

11. A process according to claim 1, wherein said NO and/or NO₂ gases are derived from off-gases produced by combustion of fossil fuels.

12. Use of off-gases produced by combustion of fossil fuels in a process according to claim 1.

13. Micro-organisms of the genera Nitrosomonas, cultured in the presence of nitrogen dioxide and nitrogen monoxide.

14. Use of micro-organisms of the genera Nitrosomonas, cultured in the presence of nitrogen dioxide and nitrogen monoxide, as activated sludge in a process for the biological treatment of ammonium-rich aqueous media.

15. A process according to claim 1, wherein the NO/NO₂ ratio is from 1:2 to 1:25 (v/v).

16. A process according to claim 1, wherein the NO/NO₂ ratio is 1:3 (v/v).

17. A process according to claim 1, wherein the ratio of NO₂ to ammonia in said media is 1:500 (v/v).

18. A process according to claim 4, wherein the NO₂ concentration in said media is 75 ppm (v/v).

19. A process according to claim 6, wherein the NO concentration in said media is 25 ppm (v/v).
